(19) United States
(12) Patent Application Publication
(10) Pub. No.: US 2006/0105028 A1
(43) Pub. Date: May 18, 2006

(54) Systems and methods for treating warts

(76) Inventors: Jie Zhang, Salt Lake City, UT (US); Kevin S. Warner, West Jordan, UT (US)

Correspondence Address: THORPE NORTH & WESTERN, LLP., 8180 SOUTH 700 EAST, SUITE 200, SANDY, UT, 84070, US

(21) Appl. No.: 11/271,020

(22) Filed: Nov. 10, 2005

Related U.S. Application Data
(60) Provisional application No. 60/627,555, filed on Nov. 12, 2004.

(51) Int. Cl. A61F 13/02; A61L 15/16

(52) U.S. Cl. 424/448

(57) ABSTRACT

The present invention is drawn to systems and methods for treating warts. The system can comprise a wart treatment formulation comprising a substance for treating warts and a cavity patch comprising an open cavity being configured to become a closed cavity upon application and adherence to a skin surface. The closed cavity can be configured to contain the wart treatment formulation.

The present application claims the benefit of U.S. Provisional Application No. 60/627,555, filed on Nov. 12, 2004, which is incorporated herein by reference in its entirety.

### FIELD OF THE INVENTION

The present invention is drawn to the treatment of warts. More particularly, the present invention is drawn to systems, methods, apparatuses, and formulations which combine multiple technologies for improved wart removal effectiveness.

### BACKGROUND OF THE INVENTION

Current treatment therapy for warts includes trimming, surgical removal, freezing with liquid nitrogen, injection with bleomycin, laser destruction, and patient applied creams containing imiquimod, and a keratolytic. The success rate of these treatments is low (typically below 60%) in large part due to poor patient compliance, as the above-mentioned procedures are painful (excepting the patient applied creams) and require multiple visits to a physicians office. The amount of pain and discomfort felt by the patients can vary depending on the type and location of the wart being treated. For example, the pain and discomfort resulting from the above procedures is usually more severe for plantar warts due to the fact that plantar warts often occur on weight bearing surfaces of the feet and toes.

Though the use of salicylic acid, imiquimod, and occlusion provides some treatment efficacy, research continues in efforts to provide treatment systems and methods which provide effective treatment with increased convenience and reduced pain.

### SUMMARY OF THE INVENTION

The present invention discloses systems and methods utilizing a closable cavity configured to contain a wart formulation to provide the benefit of continuous delivery of an active agent for treating warts, as well as provide a protective/occlusive barrier over the wart to increase efficacy and/or reduce the pain and discomfort associated certain types of warts, e.g., warts on the weight bearing surfaces of the feet and toes. Although the systems and methods of the current invention are particularly useful for treating plantar warts, they can also be used to treat other warts, including but not limited to common warts and genital warts.

In accordance with the embodiments of the present invention, a system for treating warts can comprise a wart treatment formulation comprising a substance for treating warts; and a cavity patch comprising an open cavity being configured to be closed at least in part by a skin surface. The closed cavity can be configured to contain the wart treatment formulation.

In another embodiment, a method for treating warts can comprise enclosing a wart on a skin surface with a cavity patch, wherein the cavity patch comprises an open cavity configured to be closed at least in part by a skin surface. The closed cavity can contain a wart treatment formulation which is in contact with the wart.

Additional features and advantages of the invention will be apparent from the following detailed description which illustrates, by way of example, features of the invention.

### BRIEF DESCRIPTION OF DRAWINGS

FIG. 1 is a schematic perspective view of a cavity patch in accordance with embodiments of the present invention;

FIG. 2 is a schematic perspective view of an alternative cavity patch in accordance with embodiments of the present invention; and

FIG. 3 is a schematic cross-sectional view of a cavity patch applied to a skin surface in accordance with one embodiment of the present invention.

### DETAILED DESCRIPTION OF THE PREFERRED EMBODIMENT(S)

Before particular embodiments of the present invention are disclosed and described, it is to be understood that this invention is not limited to the particular process and materials disclosed herein as such may vary to some degree. It is also to be understood that the terminology used herein is used for the purpose of describing particular embodiments only and is not intended to be limiting, as the scope of the present invention will be defined only by the appended claims and equivalents thereof.

The singular forms “a,” “an,” and, “the” include plural referents unless the context clearly dictates otherwise. Thus, for example, reference to a drug-containing composition including “a drug” includes one or more drug components and reference to “the gelling agent” includes reference to one or more gelling agents.

As used herein, “subject” refers to a mammal that may benefit from the administration of the systems or methods of this invention. Examples of subjects include humans, and may also include other animals such as horses, pigs, cattle, dogs, cats, rabbits, and aquatic mammals.

As used herein, the terms “formulation” and “composition” are used interchangeably and refer to a mixture of two or more compounds, elements, or molecules.

As used herein, the term “gelling agent” refers to a compound or compounds which, when brought into contact with a gel-triggering agent, solidify into a soft solid composition. Gelling agents are generally well known in the art as are the corresponding gel-triggering agents which cause the gelling agents to solidify.

As used herein, the term “gel-triggering agent” refers to a compound or compounds which when brought into contact with a gelling agent cause the gelling agent to form a soft solid composition. “Boric acid” or a “salt of boric acid” is exemplary of compounds that when placed into contact with a gelling agent causes the gelling agent to form a soft coherent solid.

When referring to a gel-triggering agent that is “specific for gelling” a composition containing polyvinyl alcohol, this includes any gel-triggering agent that causes a drug-containing composition that includes polyvinyl alcohol to form a soft, coherent solid upon interaction therewith. Specific non-limiting examples include boric acid, a salt of boric acid, or a borate such as sodium, lithium, or potassium borate.

The terms “gelling agent” and “gel-triggering agent” are relative terms to one another. If two compounds form a gel when contacted, then one can be considered to be a gelling agent and the other a gel-triggering agent.

The term “viscosity modifying agent” refers to substances that can increase the viscosity of the formulation and compositions of the present invention. Non-limiting examples of modifying agents include polyvinyl alcohol, ethyl cellulose, a carbomer, hydroxy propyl cellulose, a methacrylic polymer and a methacrylate polymer.

The term “coherent” refers to solids which are formed that remain substantially intact with minimal or no ripping when gently removed from a skin surface, e.g., a composition that is peelable.

As used herein, the terms “open cavity,” “drug reservoir,” “empty patch,” and “medicine cavity” refer to the interior portion of the cavity patch which is capable of containing a wart treatment formulation. In one embodiment, the wart treatment formulation can be prepared using a drug-containing composition and the gel-triggering agent to form a soft, coherent solid. The open cavity provides a retention form for the wart treatment composition, preventing the composition from spilling, leaking, or running. If using a gelling or solidification mechanism, prevention of leaking, etc., can be prior to the completion of the gelling of the wart treatment composition. The open cavity is capable of being closed at least in part by application to the skin of a subject. Generally the open cavity is defined by one or more patch wall(s) and an impermeable cover and can take on a variety of shapes and sizes. The patch wall(s) and impermeable cover can be integrated or modular. One preferred shape for the open cavity is a ring shape with an impermeable cover over one of the otherwise open ends.

The term “filled patch” refers to a cavity patch having an open cavity which has been filled with a predetermined amount of wart treatment formulation. In one embodiment, when the wart treatment formulation containing a gelling agent is placed in the open cavity, if the open cavity includes a gel-triggering agent, a soft, coherent solid can be formed.

The term “wart,” unless specified otherwise, refers to all kinds of warts, including but is not limited to plantar warts, common warts, and genital warts.

As used herein, the term “skin contact region” refers to the area of the opening of the open cavity which is to be closed by skin, or in other words, the skin contact region defines the amount of skin surface which comes into contact with the drug-containing formulation contained in the open cavity. The skin contact region is limited by the walls of the cavity patch.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity, and thus, should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. This same principle applies to ranges reciting only one numerical value. Furthermore, such an interpretation should apply regardless of the breadth of the range or the characteristics being described.

In accordance with these definitions and embodiments of the present invention, the present invention is drawn to combining multiple wart treatment schemes, each of which contributes to wart reduction or removal. These treatment schemes include a) occlusion; b) hydration; c) increased local skin temperature; and/or d) application of drug(s).

Occlusion or occlusion/hydration includes covering a wart with one or more layer(s) of an adhesive tape or bandage in order to seal the wart in a substantially watertight or airtight manner. After several hours or days, the tape or bandage can be removed and reapplied until the wart has been reduced or removed. The mechanism of why occlusion or occlusion/hydration is helpful is not entirely clear. It may be related to hydration-induced “loosening up” of the tightly packed dead cells in the thick keratinized wart surface layer. In further detail, viruses that cause warts tend to thrive in relatively cold skin areas, such as that on fingers, elbows, and ears. As such, the increasing of local skin temperature can help reduce or remove warts. In another aspect, certain drugs are also known to help cure warts. These drugs include imiquimod and keratolytics such as salicylic acid, urea (by peeling off the thick keratinized skin surface, or desquamation), to name a few. In accordance with this, the present invention is drawn to combining at least two of these treatment regimens in order to more effectively treat warts.

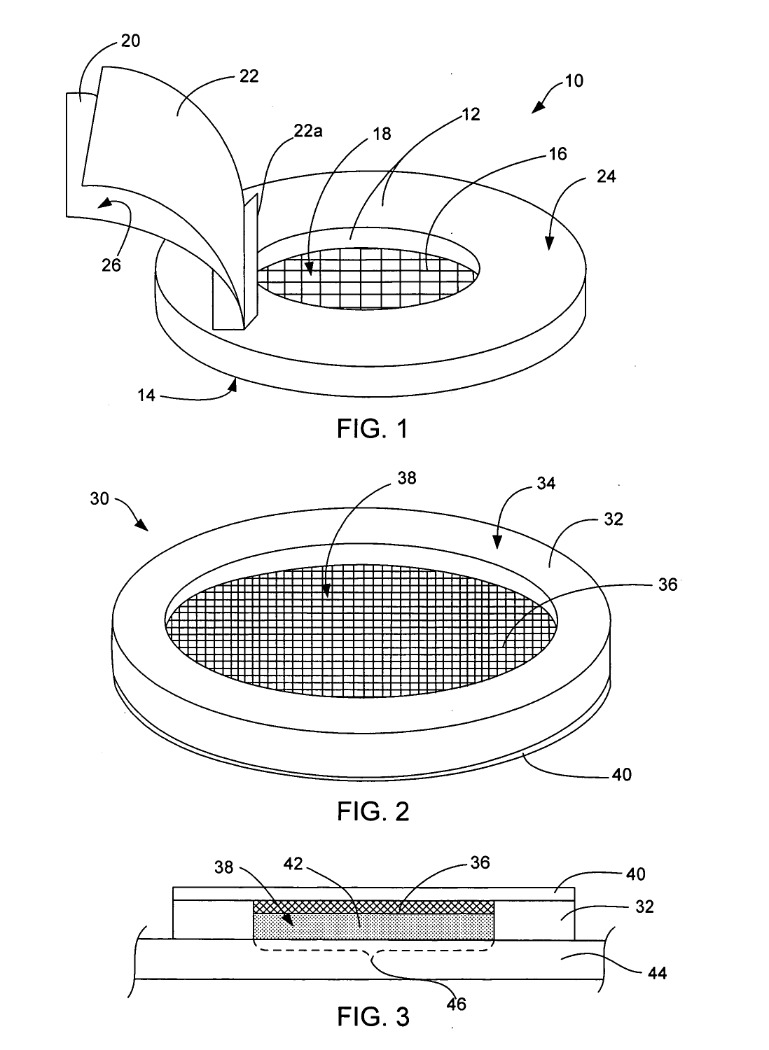

In one aspect of the invention, a cavity patch and a wart treatment formulation can be combined. The cavity patch can begin as an empty patch comprising a side walls (such as in a ring shape) with a first side completely covered with an occlusive cover, as shown in FIGS. 1-3. The other side of the ring can be coated with an adhesive for adhering the open cavity patch to the skin. The space defined by the occlusive cover and the inner space of the ring forms a cavity, which when filled, forms a “medicine cavity” or a “medicine-filled cavity.” The formulation can include wart treating compound(s) having a consistency suitable to be conveniently placed into the medicine cavity. In one embodiment with this system, the user places a predetermined amount of a wart treatment formulation into the medicine cavity and then applies the patch onto the wart such that the wart is located inside the medicine cavity, and thus, is submerged in the formulation. Alternatively, the medicine can be applied directly to the wart, and then covered by the cavity patch. In still another embodiment, the cavity walls (such as in a ring or other shape) can be applied to the skin around the wart, the cavity filled, and an occlusive backing applied to the ring to close the cavity patch, forming the medicine-filled cavity and occluding the wart.

In each of these cases, the adhesive on the ring serves to affix the patch onto the skin and seal the formulation into an enclosed space defined by the surfaces of the medicine cavity and the skin. Thus, no matter the order of application, the skin, at least in part, closes the cavity. In one embodiment, the formulation can include water for hydrating the wart, or the formulation can merely be water itself (as the active ingredient). In another embodiment, the wart treatment formulation can have a viscosity such that it does not easily spill when placed in the medicine cavity and applied to the skin. In another embodiment, the wart treatment formulation can include a viscosity modifying agent. Non-limiting examples of viscosity modifying agents include polyvinyl alcohol, ethyl cellulose, a carbomer, hydroxy propyl cellulose, a methacrylic polymer, a methacrylate polymer, and combinations thereof.

The cavity patch of the present invention has multiple effective configurations. In one configuration, the cavity patch can be top loading, as shown from a top perspective view at 10 in FIG. 1. In this embodiment, the cavity patch includes a wall 12 which is in the shape of a ring. The material of the wall can be an impermeable material, which is preferably a soft, flexible material, e.g., a closed-cell foam tape or other foam similar material. An adhesive 14 is typically coated on the wall to effectuate adhesion between the ring structure of the wall and a skin surface (not shown). In other words, the adhesive coated bottom wall is placed in direct contact with skin when the patch is in use. The cavity patch, as shown in FIG. 1, further includes an open cavity 18 (also referred to as the drug reservoir) being open on both ends like a donut to facilitate loading of a drug-containing composition into the cavity patch as well as facilitate contact between the drug-containing composition and the skin surface. One of the openings (on the bottom of the cavity patch) is substantially coextensive with the adhesive coated portion of the wall and is configured to be closed by application to the skin. The other opening (on the top of the cavity patch) is opposite the adhesive coated portion and is substantially coextensive with a top portion 24 of the wall. This opening is configured to be closed by an impermeable cover 20, preferably after application of the patch to skin and after the drug-containing composition is placed into the open cavity. The impermeable cover can include an adhesive substrate 26, and can have thermo-insulation properties in one embodiment. A release liner 22, with a leading edge 22a for gripping, is present that can be removed in order to protect the adhesive substrate until just prior to use. When the release liner is removed, the adhesive substrate of the impermeable cover can be used to close the open cavity at the top portion of the cavity patch.

The open cavity 18 is further configured to contain a gel-triggering agent. In the embodiment shown in FIG. 1, an absorbent mesh 16 impregnated or coated with the gel-triggering agent. The mesh material is used so that it does not close the open cavity, as the very nature of a mesh is that it has open areas between fibers or other material used to form the mesh. The gel-triggering agent is present which is capable of triggering a transition of a drug-containing composition (which includes the active ingredient) from a more liquid or runny state to a more solidified coherent state or soft solid gel state. The transition from the liquid state to the soft solid gel state provides the advantage that when the patch is removed, the composition leaves little to preferably no residue on the skin. Thus, in this embodiment the wart treatment formulation is formed as the gel-triggering agent and the gelling agent of the drug-containing composition form a soft, coherent solid within the patch.

To use the cavity patch shown in FIG. 1, the user adheres the cavity patch 10 to a skin surface with the adhesive 14 contacting the skin. The subject then places the drug-containing composition into the open cavity 18, and then closes or seals the open cavity with the impermeable adhesive cover 20 from which the release liner 22 has been removed. The gel triggering agent on or impregnated within the mesh 16 diffuses into the drug-containing composition and causes the composition as a whole to gel into a soft solid over a period of time, thus forming a solidified patch including the final wart treatment formulation that can be removed after desired application without leaving residue from the drug-containing composition on the skin. In accordance with embodiments of the present invention, the gel-triggering agent and the drug-containing composition are positioned in the system in such a way that they are kept isolated from one another until immediately before or during use of the cavity patch. In another embodiment, if a gel-triggering agent is not used, then the wart treatment formulation in its immediately useable form (from a tube or other dispenser) can be placed directly into the cavity patch on the skin and then closed by applying the occlusive backing.

In an alternative configuration, the cavity patch 30 can be bottom loading, as shown from a bottom perspective view at in FIG. 2. A bottom portion of the ring shaped patch wall 32 is coated with an adhesive 34. The cavity patch has an open cavity 38 which is configured to be closed by application to a skin surface (not shown). Unlike the top loading cavity patch of FIG. 1, the bottom loading cavity patch of this embodiment has only one opening, the opening being coextensive with the bottom portion of the patch wall and the adhesive. The open cavity is defined by the patch wall and an impermeable cover 40 which is substantially coextensive with a top portion of the patch wall. Optionally, inside of the open cavity is a mesh 36 which is coated or impregnated with a gel-triggering agent. In this embodiment, the mesh can be attached to the impermeable cover, or can be suspended at any location within the open cavity.

In accordance with FIG. 2, to use the device, the user places the drug-containing composition (which can be the wart treatment formulation or a formulation that becomes the ultimate wart treatment formulation upon interaction with the gel-triggering agent, depending on whether a gelling mechanism is used or not) into the open cavity 38 of the cavity patch 30 with the bottom (adhesive side) up. When the drug-containing composition is loaded in the open cavity, the patch is placed onto the skin so that the adhesive 34 becomes affixed to the skin. The drug-containing composition (or wart treatment formulation) is at least substantially sealed in the now closed open cavity by the impermeable cover 40, the patch wall 32 and the skin surface. If using the gel-triggering agent, the gel-triggering agent in the mesh diffuses into the drug-containing composition and gels it into a soft solid after a certain amount of time, thus forming a solidified patch that can be removed after the desired application without leaving residue from the drug-containing composition on the skin. As mentioned above with respect to FIG. 1, in this embodiment as well, the gel-triggering agent and the drug-containing composition are positioned in the system in such a way that they can be kept isolated from one another until immediately before or during use of the cavity patch. When the drug administration period is complete, the patch can be removed from the skin and leave no substantial gel residue, if any, on the skin. As mentioned, if a gelling mechanism is not being used, then the wart treatment formulation can be applied into the cavity patch from a dispensing device and applied to the skin in a similar manner. It should be noted that in embodiments where gelling is not necessary at the time of application, cavity patches can be filled by the user, or can be pre-filled and pre-packaged by the manufacturer.

Turning to FIG. 3, a cross-sectional view of the cavity patch 30 of FIG. 2 is shown applied to a skin site 44. As mentioned above, this embodiment includes an impermeable cover 40, a patch wall 32, an open cavity 38 (which is shown as closed by the skin site), and a mesh or non-woven material 36 carrying a gel-triggering agent. However, in the embodiment shown, the drug containing composition 42 is loaded in the cavity (for interaction with the skin and the gel-triggering agent). The skin site includes a skin contact region 46 where the drug-containing composition contacts the skin surface. It should be noted that in the top loading system shown in FIG. 1, the skin contact region is found between the fibers of the mesh.

It should be noted that as warts often grow in contoured skin surfaces, the cavity patches in accordance with embodiments of the present invention can be made of a flexible and/or soft material, and the adhesive can be of sufficient tackiness so that the filled patch can be conveniently and securely affixed onto the contoured skin surfaces for the entire duration of treatment.

The filled patch can also provide insulation to the local skin, especially when the occlusive cover is made of an insulating material, such as foam tape. Alternatively, heating devices can be used to heat the skin and/or the cavity patch (including the drug-containing formulation/gel-triggering agent) to achieve enhanced results related to drug absorption, drug delivery from a depot beneath the skin, reducing onset time, or other benefits related to adding heat to a transdermal delivery system. Exemplary heating devices and appropriate uses for such heating devices that can be used in accordance with embodiments of the present invention are described in U.S. Pat. Nos. 6,955,819, 6,780,426, 6,756,053, 6,726,673, 6,613,350, 6,546,281, 6,488,959, 6,465,006, 6,453,648, 6,340,472, 6,306,431, 6,303,142, 6,284,266, 6,261,595, 6,245,347, and 5,919,479, each of which are incorporated herein by reference to the extent they are compatible with embodiments of the present invention.

Preferably, the wart treatment formulation can include an appropriate drug for treating or removing warts. For example, salicylic acid and/or urea can be incorporated into a wart treatment formulation. Unlike traditional formulations and associated methods, the present invention provides means to occlude the formulation in the medicine cavity for essentially the entire duration of application. Thus, the hydration effect in combination with the occlusion allows more drug to penetrate into the wart tissue, increasing the effectiveness of the treatment. Another drug that would benefit from this system is imiquimod. The commercially available imiquimod product, Aldara (3M), is not typically effective in treating plantar warts or warts on regular skin (as opposed to genital warts) if it is rubbed in the skin (currently most common method of use). One explanation might be that that Aldara is transferred to clothing or objects that often come into contact with the skin shortly after it is rubbed in the skin. The present invention addresses this problem by including an occlusive barrier to ensure the formulation and the solvent vehicle remains in contact with the skin as described herein.

In accordance with the embodiments described herein, an apparatus can comprise a cavity patch having an open cavity, the open cavity defined by: i) a ring or ridge of another shape having a predetermined thickness and a predetermined inner volume, ii) a layer of adhesive coated on a ring of the ring or ridge on a first side, and iii) an occlusive cover attached to a second opposing side of the ring or ridge, thus sealing the opening of the ring or ridge thereto. Though the ring structure and the occlusive cover are described above as two separate entities that are adhered together, it is understood that the ring structure and the occlusive cover can be a single structure of the same material, e.g., rubber or other flexible or moldable materials. Whether using a single integrated material or multiple materials adhered together, this configuration provides a cavity or space defined by the inner volume of the ring or ridge and the occlusive cover. Further, other shapes rather than ring-shaped cavity patches can be used, e.g., dome-shaped, rectangular shaped, etc.

As discussed above, each open cavity is configured so as to be closed at least in part by application to a skin surface. The region of skin that closes off at least in part the open cavity is referred to as the skin contact region. The area or size of the skin contact region can be varied according to the needs of the desired application, or more particularly the size or number of warts being treated. For example, for treating a single wart, an area of 0.1 to 1 cm² may be appropriate. Depending on the application and/or treatment regimen, the skin contact region can have an area of from 0.1 and 20 cm², and preferably from 0.2 to 5 cm² and even more preferably 0.1 to 2 cm². The open cavity can have a thickness or depth approximately equivalent to the thickness or depth of the cavity patch. The thickness (depth) of the open cavity can also be relevant to the performance of the system. If the open cavity is too shallow, the volume of the drug-containing composition that can be accommodated by the open cavity might be insufficient to ensure complete skin coverage with the drug-containing composition. If the depth of the open cavity is too great, it may take too long of a time for the gel-triggering agent to diffuse throughout the drug-containing composition to gel substantially the entire composition into a soft solid. In one embodiment, the depth of the open cavity can be from 0.1 mm to 5 mm, and more preferably between 0.2 mm to 3 mm.

Within the cavity or space, a wart treatment formulation can be present, which comprises a substance for treating warts, and optionally, a gelling agent. The wart treatment formulation can have a consistency so that it can be conveniently placed into the medicine cavity without spilling, e.g., gel, ointment, lotion, cream, etc. Alternatively or additionally, heat can be applied to the cavity patch, whether or not hydration or other wart treatment formulation is also used.

As mentioned, the cavity patch and the wart treatment formulation can both be similar to that of the basic systems described above, except that a gel-triggering agent is coated inside the medicine cavity, i.e. on the inside wall of the ring and/or on the occlusive cover or another layer of material, rather than on a separate structure, e.g., gauze, non-woven, mesh material, etc. The wart treatment formulation can include a matching gelling agent that, when brought into contact with a gel-triggering agent, can convert the formulation from a viscous fluid or a semisolid state to a soft, coherent solid. With respect to this embodiment, the formulation changes into a soft, coherent solid within a certain time after it is placed into the medicine cavity, e.g., upon being brought into contact with the gel-triggering agent. When the patient removes the patch after the application, the solidified formulation can be formulated such that it does not leave a residue on the skin.

An example of acceptable gelling agent and gel-triggering agent is polyvinyl alcohol and boric acid (or a salt of boric acid or a borate), respectively. For example, if the medicine cavity with a thickness of 1 mm and contains 10 mg/cm² of sodium borate, i.e. coated on the occlusive cover and/or inner surface of ring, and the formulation is a water-based viscous fluid containing 10 wt % polyvinyl alcohol, the formulation can be converted into a soft, coherent solid within 10 to 120 minutes after being placed into the medicine cavity. In another embodiment, boric acid (or salt of boric acid or borate) can be impregnated into an absorbent material at a quantity from 1 to 20 mg/cm². In another embodiment, the amount of boric acid or the salt of boric acid impregnated into an absorbent material and disposed within the open cavity of the cavity patch can be from 4 to 8 mg/cm².

In these or other embodiments, the wart treating medicine or substance of the wart treating formulation can be water (for its softening action on keratinized wart surface); compounds capable of loosening and/or thinning keratinized skin surfaces such as keratolytics, e.g., salicylic acid; alpha hydroxy acids, e.g., citric acid, lactic acid, malic acid, tartaric acid, or glycolic acid; urea; benzoyl peroxide; tretinoin; sulfur; rescorinol; trichloroacetic acid; imiquimod; or a combination of the above. In one embodiment, a formulation comprising water, a keratolytic agent, and a wart treatment compound, e.g., imiquimod, can be especially effective, as those ingredients may work additively, cumulatively, or even synergistically.

As a further note, the formation of the water-based formulation into a soft, coherent solid after being placed into an open cavity patch can include a delicate balance between amount of gel-triggering agent, e.g., sodium borate, cavity thickness, gelling agent concentration, e.g., polyvinyl alcohol, composition or solution pH, and viscosity. Using boric acid and polyvinyl alcohol as an example, the boric acid (gel-triggering agent) provides for the gelling of the drug-containing composition. If the boric acid concentration is too low, the driving force for boric acid diffusion across the drug formulation in the cavity is reduced because of the lower concentration of borate ions dissolved at the aqueous formulation/nonwoven fabric interface. Cavity thickness can also impact the length of time the borate ions diffuse throughout the formulation in the cavity to gel the formulation into a solid. Increased concentrations of polyvinyl alcohol can increase the time for borate ions to diffuse throughout an increasingly viscous formulation in the cavity to gel the formulation as a whole, and “consume” borate ions in the process. Additionally, molecular weight for polyvinyl alcohol can also affect the gelling properties. Polyvinyl alcohol (PVA), USP (U.S. Pharmacopia), has a molecular weight from about 30,000 Mw to 50,000 Mw, and thus, the Examples provided utilize PVA within this range. However, molecular weights outside of this range also can form acceptable soft, coherent solids in accordance with embodiments of the present invention. Further, solution pH below 5 can result in incomplete solidification of the formulation because it is believed that the hydrogen ion content is high enough to compete with the polyvinyl alcohol in the complexation reaction with borate ions. Though certain parameters have been discovered as preferred, any combination of drug-containing composition and gel-triggering agent that works in the context of use in a cavity patch in accordance with embodiments of the present invention is included herein. This being stated, the impact of each of the above-mentioned parameters are explored in the Examples to provide the best known compositions, methods, and systems of practicing the present invention.

### EXAMPLES

The following examples illustrate the embodiments of the invention that are presently best known. However, it is to be understood that the following is only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what is presently deemed to be the most practical and preferred embodiments of the invention.

### Example 1

A cavity patch is prepared having a configuration similar to that in FIG. 1 (top loading). A drug-containing composition (solution) containing an active drug and 15% polyvinyl alcohol in water. The cavity patch contains an absorbent mesh material which is a thin layer of absorbent gauze impregnated with about 5 mg/cm² sodium borate, and which is adhered on the underside of the ring-shaped patch wall (made from a soft, flexible foam tape) such that the mesh covers, but does not close, the lower end of the cavity in the ring. An impermeable cover is made of an impermeable tape (such as the 1525 adhesive tape by 3M) with a release liner covering the adhesive side.

To use the system, the user first affixes the cavity patch onto the skin so that the adhesive layer firmly seals the bottom of the patch onto the skin. Eight one hundredth of a milliliter of the drug-containing composition is dispensed onto the mesh and spreads over the skin area defined by the cavity. The user then pulls the release liner on the impermeable cover horizontally to seal the upper end of the open cavity with the cover. The solution is now sealed in the space defined by the skin, the cover, and the walls defining the once open cavity. After 20-30 minutes, the solution forms a soft, coherent solid gel while it is delivering drug into the skin, into the surrounding tissue, or into systemic circulation. The drug is delivered to the user's body continuously before and after the formulation is gelled. When the intended administration period is over, the entire loaded patch is simply removed form the skin. No residue from the drug-containing composition is left on the skin when the patch is removed since the drug-containing composition has gelled into a soft solid.

### Example 2

The drug-containing composition (solution) and materials in the cavity patch are the same as those in Example 1, but the configuration of the cavity patch is the bottom-loading type as shown in FIG. 2. An amount of drug-containing composition is dispensed into the open cavity of the cavity patch prior to adhesion of the patch to the skin. After the drug-containing solution is dispensed into the open cavity, the user affixes the cavity patch to the skin using the adhesive layer which is coated on the bottom of the cavity patch wall. This seals the patch to the skin and seals the drug-containing composition in the open cavity. The drug-containing composition gels into a soft solid and leaves no residue when the cavity patch and gel are removed at the end of the intended administration period.

### Example 3

Four cavity patches having the configuration as shown in FIG. 2 and containing 0.5, 1, 2, and 4 mg/cm² borate are studied to determine an optimal amount of borate (a gel-triggering agent) needed to gel a 15% polyvinyl alcohol (PVA) (a gelling agent) solution. The open cavity is 3 mm deep by 14 mm in diameter, and the absorbent material is a thin layer of nonwoven film impregnated with borate. The nonwoven material is affixed to the underside of the impermeable cover (3M 1523 polyethylene film) such that the nonwoven film covers the entire area of the open cavity. Specifically, the cavity patches containing 0.5, 1, 2, and 4 mg/cm² borate are each dosed with approximately 0.5 mL of a 15% PVA in water formulation, so that the filled open cavity has roughly 50 mg PVA per cm². The borate to PVA ratios per unit area for the patches are approximately 1:100, 2:100, 4:100 and 8:100, respectively. Each patch is then affixed to the upper arm of a study volunteer. After 3 hours, one side of the patch is lifted off the skin to observe the extent of PVA gelling in the open cavity. Only the patch containing 4 mg/cm² borate is gelled, this patch is then completely removed and there is no residue remaining on the arm. After 5 hours the remaining three patches are observed again, and the patch containing 2 mg/cm² borate has mostly gelled, upon removal of this patch, a thin layer of PVA solution the diameter of the skin contact region remains on the arm. The patches containing 0.5 and 1 mg/cm² borate are not gelled and are removed.

Although the patches containing 0.5 and 1 mg/cm² borate do not gel within 5 hours, they may gel after longer waiting times. However, prolonged gelling time increases the risk of spilling and thus is less desirable. Therefore, it is preferable to have sufficient amount of borate and appropriate concentrations of PVA in the system (see examples below) so that the formulation is gelled within 5 hours, and most preferably within 2 hours. From the data of this example, the ratio of borate to PVA is preferably greater than 2:100, more preferably greater than 4:100, and most preferably greater than 8:100, in order to achieve reasonably short gelling times.

As exemplified, an appropriate amount of borate impregnated in the nonwoven material can be used to ensure complete or substantially complete gelling of the drug formulations during the intended application time. The observed delay in gelling or lack of gelling of the PVA in the patches with borate concentrations below 2 mg/cm² can be explained by two factors: 1) there is not enough free borate available to undergo the interaction with the PVA, and 2) the driving force for borate diffusion across the open cavity is reduced because of the lower concentration of borate ions dissolved at the aqueous formulation/nonwoven fabric interface.

### Example 4

Three cavity patches with 14 mm diameter open cavities of different depths are prepared. The three depths are 0.13 mm, 0.26 mm, 1 mm, and 3 mm, respectively. A borate concentration of 4 mg/cm² is placed in each cavity patch. The cavity patches used are similar to that shown in FIG. 2 (bottom loading). The nonwoven material is affixed to the underside of the impermeable cover (3M 1523 polyethylene film) such that the nonwoven film covers the entire area of the open cavity. The purpose of this example is to study the length of time needed to gel a 25% PVA in water solution in each of the patches.

Each of the 4 patches is affixed to the upper arm of a study volunteer. After 30 minutes, one side of the patch is lifted off the skin to observe the extent of PVA gelling in the open cavity. After 30 minutes, the compositions in the patches with 0.13 mm and 0.26 mm deep open cavity patches are gelled. The patches are then removed. After 1.5 hours the 1 mm deep open cavity is gelled. After 3 hours the composition in the patch with a 3 mm deep open cavity is gelled.

The depth of the open cavity in the cavity patch has an impact on the time for a formulation to gel. Complexation of PVA in an aqueous solution in the presence of borate ions is dependent on the dissolution and diffusion of borate from the nonwoven material into the PVA solution. The longer gelling time for the 3 mm patch configuration may be due to the length of time required for the borate to diffuse throughout the formulation in the cavity to gel the formulation into a soft solid. Therefore, although formulations filled in open cavities deeper than 3 mm may still gel after long waiting time, such patch depth might be undesirable in some circumstances, as it increases the risk of spilling in short duration uses. Such patch depths may be practical in situations where longer uses are desirable.

### Example 5

A cavity patch similar to that shown in FIG. 2 (bottom loading) with a 3 mm depth and a 14 mm diameter having a borate concentration of 4 mg/cm² is studied to determine the length of time needed to gel 15%, 25%, and 40% PVA in water formulations. Again, the nonwoven material is affixed to the underside of the impermeable cover (3M 1523 polyethylene film) such that the nonwoven film covers the entire area of the open cavity. Each of the formulations is dosed into a cavity patch and is then affixed to the upper arm of a study volunteer. After 2, 3, 4, and 5 hours, one side of the patch is lifted off the skin to observe the extent of PVA gelling in the open cavity. The 15% PVA in water formulation is gelled at 2 hours, the 25% PVA formulation gelled at 3 hours, and the 40% PVA formulation had not gelled after 5 hours of wear. In each instance, upon removal of the patch after gelling was observed, no formulation residue is observed on the skin.

The concentration of PVA in an aqueous solution impacts the gelling time. Complexation of PVA in an aqueous solution in the presence of borate ions is dependent on the dissolution and diffusion of borate from the nonwoven material into the PVA solution. The longer gelling time as a function of increased PVA concentration is due to the length of time required for the borate to diffuse throughout an increasingly viscous formulation in the open cavity to gel the formulation into a solid, and “consumption” of the borate ions by PVA during the diffusion process. On the other hand, formulations with PVA concentrations lower than 8%, and especially those lower than 5%, tend to have difficulty solidifying into a solid with satisfactory strength. Therefore, the concentration of PVA in accordance with embodiments of the present invention is preferably in the range of 5 to 40%, and more preferably in the range of 8 to 30%.

### Example 6

A cavity patch having a configuration similar to that in FIG. 2 is prepared. Two placebo drug-containing compositions are studied. Composition A contains 12% PVA, 19% ISA, 2% trolamine, and 0.3% TR-2 (an emulsifier), and 66.7% water, with a pH of 7. Composition B contains 12% PVA, 19% ISA, 0.3% TR-2 (an emulsifier), and 68.7% water with a pH of 5.5. The open cavity in each of the two cavity patches is 3 mm deep with a 14 mm diameter, and the absorbent material is a thin layer of nonwoven film impregnated with 5 mg/cm² of borate. The nonwoven film is affixed to the underside of the impermeable cover (3M 1523 polyethylene film) such that the nonwoven film covers the entire area of the open cavity.

The system is used by dosing approximately 0.5 g of each placebo drug-containing composition into the open cavity, removing the release liner from the adhesive layer, and affixing the adhesive layer quickly to the skin. After 5 hours, the patches are evaluated to determine if the compositions have gelled into a solid. Composition A forms a soft solid gel and upon removal, there is no residue from the composition remaining on the skin. On the other hand, composition B does not form a gel after 5 hours. The patch is inspected again after 7 hours, and the formulation still has not formed a solid.

These results indicated that the gelling process of PVA in the presence of borate is dependent on pH. An optimal pH range for PVA gelling in the presence of borate is 6-9 (pHs higher than 9 may cause skin irritation, though these pH levels are still workable). Without being bound by any particular theory, at pH values lower than 5, the hydrogen ion concentration may be high enough to compete with PVA in the complexation reaction with borate ions.

### Example 7

A cavity patch similar to that shown in FIG. 2 (bottom loading) is prepared. An active drug-containing composition (Composition A) containing 12% PVA, 19% ISA, 2% trolamine, 4% imiquimod, 0.3% TR-2 (an emulsifier), and 62.7% water is dosed into the open cavity (3 mm deep and 14 mm diameter). The mesh coated material is a nonwoven impregnated with 4 mg/cm² borate as the gel triggering agent. The impermeable cover was a 3M 1523 polyethylene film, and the open cavity is formed with a foam tape. Approximately 0.5 g of drug solution is dosed into the open cavity. A release liner is removed exposing the adhesive layer on the bottom of the patch and the patch is immediately affixed to hairless mouse skin (HMS) previously mounted on a Franz diffusion cell. After approximately 4 hours, the patch is removed and the drug solution in the open cavity has formed a solid gel. No residue is left on the skin and the drug solution has not migrated from the area defined by the foam tape.

### Example 8

The formulation described in Example 7 is compared to Aldara (3M) for average skin flux in a hairless mouse skin (HMS) in vitro model. The HMS is mounted carefully between the donor and receiver chambers of a Franz diffusion cell. The receiver chamber is filled with pH 7.4 phosphate buffered saline (PBS). The experiment is initiated by placing the Example 7 formulation noted above in the open cavity (with 5 mg/cm² sodium borate) on the stratum corneum (SC) side of the skin sample. The Aldara is applied without sodium borate as directed. Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time.

Table 1 shows data obtained using the experimental process outlined above.

TABLE 1
Steady-state flux (J) of Imiquimod through HMS

| | J |
|---|---|
| Formulation | (μg/cm²/h)* |
| Aldara | 0.21 ± 0.03 |
| Example 7 gelled with Na Borate | 0.32 ± 0.09 |

*Flux value is the average and standard deviation of three determinations.

### Example 9

The formulations, materials, and experimental design are the same as in Examples 2-7, but the configuration of the cavity patch is the top loading type as shown in FIG. 1. Due to the configuration of the cavity patch, drug-containing solution is placed into the open cavity after the cavity patch is affixed to the skin as taught in Example 1.

### Examples 10-13

Several cavity patches with a gel-triggering agent impregnated therein are prepared in accordance with embodiments of the present invention (top loading or bottom loading), and four drug-containing compositions are prepared in accordance with Table 2, as follows:

TABLE 2

Example

| | | | |
|---|---|---|---|
| 10 | 11 | 12 | 13 |

% by Weight

| | | | | |
|---|---|---|---|---|
| Polyvinyl alcohol (PVA) | 10.9 | 11.1 | 10.7 | 10.8 |
| Water | 61.8 | 62.9 | 60.5 | 61.2 |
| Ultrez 10 | 0.1 | 0.5 | 0.3 | 0.05 |
| Isostearic acid | 18.9 | 18.5 | 21.7 | 19.1 |
| Pemulen TR-2 | 0.3 | 0.3 | 0.3 | 0.3 |
| Trolamine | 3.6 | 2.3 | 2.1 | 4.25 |
| Imiquimod | 4.4 | 4.4 | 4.4 | 4.3 |

In each of Examples 10-13, the materials in Table 2 are combined according to the following procedure. The polyvinyl alcohol (PVA) and water are combined in a glass jar and heated with stirring until the PVA has dissolved. The Ultrez 10 is added to the PVA/water mixture and the mixture is stirred until the Ultrez dissolves into the solution. The isostearic acid and Pemulen TR-2 are then added to the solution and the mixture is stirred until the TR-2 is dissolved and a white solution is formed. Trolamine and imiquimod are then added and the entire mixture is vigorously mixed at room temperature.

### Examples 14-15

Two cavity patches with a gel-triggering agent impregnated therein are prepared in accordance with embodiments of the present invention (top loading or bottom loading), and two drug-containing compositions are prepared in accordance with Table 3, as follows:

TABLE 3

Example

| | |
|---|---|
| 14 | 15 |

% by Weight

| | | |
|---|---|---|
| Polyvinyl alcohol | 11.1 | 11.1 |
| Water | 62.7 | 63.2 |
| Carbopol 980 | 0.9 | 0.1 |
| Isostearic acid | 18.3 | 18.7 |
| Pemulen TR-2 | 0.3 | 0.3 |
| Trolamine | 2.2 | 2.2 |
| Imiquimod | 4.5 | 4.4 |

In each of Examples 14-15, the materials in Table 3 are combined according to the following procedure. The polyvinyl alcohol (PVA) and water are combined in a glass jar and heated with stirring until the PVA has dissolved. Carbopol 980 is added to the PVA/water mixture and the mixture is stirred until the Carbopol dissolves into the solution. The isostearic acid and Pemulen TR-2 are then added to the solution and the mixture is stirred until the TR-2 is dissolved and a white solution is formed. Trolamine and imiquimod are then added and the entire mixture is vigorously mixed at room temperature.

### Example 16-17

Two cavity patches with a gel-triggering agent impregnated therein are prepared in accordance with embodiments of the present invention (top loading or bottom loading), and two drug-containing compositions are prepared in accordance with Table 4, as follows:

TABLE 4

Example

| | |
|---|---|
| 16 | 17 |

% by Weight

| | | |
|---|---|---|
| Polyvinyl alcohol | 11.0 | 11.2 |
| Water | 62.28 | 63.2 |
| Carbopol 981 | 0.9 | 0.1 |
| Isostearic acid | 19.0 | 18.5 |
| Pemulen TR-2 | 0.22 | 0.3 |
| Trolamine | 1.9 | 2.2 |
| Imiquimod | 4.7 | 4.5 |

In each of Examples 16-17, the materials in Table 4 are combined according to the following procedure. The polyvinyl alcohol (PVA) and water are combined in a glass jar and heated with stirring until the PVA has dissolved. Carbopol 981 is added to the PVA/water mixture and the mixture is stirred until the Carbopol dissolves into the solution. The isostearic acid and Pemulen TR-2 are then added to the solution and the mixture is stirred until the TR-2 is dissolved and a white solution is formed. Trolamine and imiquimod are then added and the entire mixture is vigorously mixed at room temperature.

### Example 18

A cavity patch with a gel-triggering agent impregnated therein is prepared in accordance with embodiments of the present invention (top loading or bottom loading), and a drug-containing compositions is prepared in accordance with Table 5, which included a keratolytic agent (benzoyl peroxide):

TABLE 5

| | Example |
|---|---|
| | 18 |
| | % by Weight |
| Polyvinyl alcohol | 11.0 |
| Water | 62.22 |
| Ultrez 10 | 0.05 |
| Isostearic acid | 18.0 |
| Pemulen TR-2 | 0.2 |
| Trolamine | 2.04 |
| Benzoyl Peroxide | 2.8 |
| Imiquimod | 3.7 |

The materials in Table 5 are combined according to the following procedure. The polyvinyl alcohol (PVA) and water are combined in a glass jar and heated with stirring until the PVA had dissolved. The isostearic acid and Pemulen TR-2 are then added to the PVA/water solution and the mixture is stirred until the TR-2 is dissolved and a white solution is formed. Benzoyl peroxide is added and then enough trolamine is added dropwise until the pH is 6.5-7.0. The entire mixture is then vigorously mixed at room temperature.

### Example 19

The formulations of Examples 10-13, 15, 17, and 18 are tested in a human cadaver skin in vitro model. Human epidermal membrane (HEM) is used as the model membrane for the in vitro flux studies described herein. The HEM is mounted carefully between the donor and receiver chambers of a Franz diffusion cell. The receiver chamber is filled with pH 7.4 phosphate buffered saline (PBS).

The experiment is initiated by placing each of the test formulations (10-13, 15, 17, and 18) in an open cavity having 5 mg/cm² sodium borate impregnated on a non-woven fabric (bottom loading) on the stratum corneum (SC) of the skin sample. For comparison purposes, a product known as Aldara (3M) which is often used to treat genital warts, is also tested (without using a cavity patch). Franz cells are placed in a heating block maintained at 37° C. and the HMS temperature is maintained at 35° C. At predetermined time intervals, 800 μL aliquots are withdrawn and replaced with fresh PBS solution. Skin flux (μg/cm²/h) is determined from the steady-state slope of a plot of the cumulative amount of permeation versus time.

Table 6 shows data obtained using the experimental process outlined above.

TABLE 6
Steady-state flux (J) of Imiquimod through HEM

| | J |
|---|---|
| Formulation | (μg/cm²/h) |
| Example 10 gelled with Na Borate | 1.1 ± 0.5 |
| Example 11 gelled with Na Borate | 1.7 ± 0.3 |
| Example 12 gelled with Na Borate | 1.4 ± 0.09 |
| Example 13 gelled with Na Borate | 0.2 ± 0.08 |
| Example 15 gelled with Na Borate | 2 ± 2 |
| Example 17 gelled with Na Borate | 0.2 ± 0.03 |
| Example 18 gelled with Na Borate | 0.1 ± 0.07 |

The formulations of the invention shown above generally provide for significant penetration of the active ingredient, and further, these values were found to be comparable to the marketed product Aldara.

### Example 20

Viscosity values reported below are obtained by conventional method used by those skilled in the art. The measurement of the viscosity of the example formulations are compared to a control sample containing only polyvinyl alcohol (PVA) and water and are run under the same conditions. Comparison to the control is illustrative. The viscosity values are obtained using a Brookfield RVDV-1+viscometer with an S-15 spindle (viscosity values reported at 2 rpm and at 25 C.). The following examples are provided to illustrate advantages of certain embodiments of the present invention, but are not intended to be limiting thereof.

TABLE 7
Viscosity values of Examples

| Example | Viscosity @ 2 rpm |
|---|---|
| 10 | 69500 |
| 11 | 20250 |
| 12 | 27750 |
| 13 | 32500 |
| 14 | 55000 |
| 15 | 30000 |
| 17 | 12500 |
| 18 | 7250 |
| Control* | 7350 |

*Control patch consisted of all the components in Example 10 except the thickening agent (Ultrez 10, Carbopol 980, or Carbopol 981) was not included.

The formulations of Table 7 all had sufficient viscosity such that the compositions did not spill when placed in the empty patch cavity and applied to a skin surface.

### Example 21

A circular ring is cut out of a medical grade foam tape (3M 1779), which has a thickness of 1/16 inch. The ring's inner and outer diameters are ⅜ inch and ¾ inch, respectively. A gauze disc with a diameter of ½ inch is impregnated with 10 mg sodium borate (gel-triggering agent). A disc with a diameter of ¾ inch is cut out of another medical grade foam tape with a thickness of 1/32 inch (3M9773). The ring, the gauze disc, and the foam tape disc are co-centrally assembled into an open cavity patch configuration such that the foam tape disc completely covers the non-adhesive side of the ring, and the gauze disc is sandwiched between the ring and the foam tape disc. The ring's adhesive side is thus still open, and the gauze is visible from the opening. The space defined by the inner space of the ring and the gauzed lined foam tape disc is the medicine cavity. The cavity patch is placed on a release liner with the adhesive side resting on the release liner (with multiple cavity patches), similar to that shown in FIG. 2.

Separately, an oil-in-water viscous fluid formulation (wart treatment formulation) is then prepared which includes 3 wt % imiquimod, 10 wt % benzoyl peroxide, 15 wt % polyvinyl alcohol (gelling agent), 0.5 wt % Carbopol 981 (thickening agent), 0.2 wt % sodium hydroxide (agent for neutralizing Carbopol 974), 10 wt % petrolatum (oil phase), 5 wt % stearyl alcohol (oil phase), 5 wt % polysorbate (emulsifying agent), and 51.3 wt % water and is loaded into a standard 20 mL aluminum ointment tube with a screw cap.

To use, the cavity patch is removed from the release liner, which exposes the opening of the medicine cavity. About 0.1 mL of the wart treatment formulation is placed in the medicine cavity, and the filled patch is covered over the wart. In this configuration, the wart becomes essentially completely submerged in the formulation, and the patch is firmly secured on the skin via adhesive on the ring. During the treatment, water occluded in the medicine cavity hydrates the wart surface for the duration that the patch is on the wart, the salicylic acid performs its function of loosening the keratin and desquamation, and imiquimod activates the local immune system to fight against the wart virus. After a predetermined time period, e.g., 12 hours, 24 hours, or more, the patch is removed from the wart. Since the formulation has long been gelled into a soft, coherent solid, substantially no residual formulation is left on the skin. The above process may be repeated over and over again until a desired effect is achieved.

### Example 22

A cavity patch is prepared as described in Example 21, except that the mesh impregnated with sodium borate is fastened to the ring, and an occlusive membrane with one side coated with an adhesive and covered with a release liner is on a non-adhesive side of the ring. In other words, the ring is applied to the skin, the medicine is applied to the wart within the ring, and the occlusive membrane is applied to the ring, enclosing the wart within the filled cavity, similar to that shown in FIG. 1.

In this embodiment, the wart treatment formulation is the same, except that the polyvinyl alcohol concentration is 10 wt % and the water content is 56.1 wt % (less viscous than that in Example 21). To use the system, the user places the cavity patch (without the occlusive backing adhered thereto) on the wart so that the entire wart is inside the ring. Thus, the adhesive coated on the bottom of the ring affixes and seals the bottom of the ring on the skin. The wart treatment formulation is placed in the medicine cavity, and the release liner on the occlusive membrane is pulled horizontally, guiding the occlusive backing onto the ring structure as the release liner is pulled away. The adhesive on the occlusive membrane seals the medicine cavity. Alternatively, the occlusive backing/release liner can be a completely separate structure than the ring, where the release liner is removed from the occlusive backing, and the occlusive backing is carefully placed on the ring to ensure a substantially air tight seal within the medicine cavity.

### Example 23

The present system includes three elements: a wart treatment formulation, a cavity patch, and a heating unit. The wart treatment formulation and the cavity patch are the same as that in Example 22 or Example 23. The heating unit is a iron oxidation based device described in U.S. Pat. No. 6,453,648. The heating unit has an area approximately the size of the cavity patch (or slightly larger), and is placed on top of the occlusive membrane either as an integral part of the cavity patch or as independent unit that can be placed on the occlusive membrane (which can be separately adhered thereto). The heat increases local skin temperature by a few degrees, e.g., to about 37° C. from typical 32° C.) which not only helps control the wart virus (as this virus prefers cooler temperatures), but also increases the hydration effect and permeation flux of the drug(s) into the wart surface.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A system for treating warts, comprising:
a wart treatment formulation comprising a substance for treating warts; and
a cavity patch comprising an open cavity being configured to become a closed cavity at least in part by application and adherence to a skin surface, said closed cavity configured to contain the wart treatment formulation.

2. The system of claim 1, wherein the open cavity is defined by an occlusive cover and a patch wall attached to the occlusive cover.

3. The system of claim 1, wherein the wart treatment formulation has a viscosity such that it can be conveniently placed into said open cavity without spilling.

4. The system of claim 1, wherein the wart treatment formulation further includes a gelling agent.

5. The system of claim 2, wherein the patch wall is a ring.

6. The system of claim 2, wherein said occlusive cover is medical tape.

7. The system of claim 2, wherein said occlusive cover is foam tape having a thermo insulation property.

8. The system of claim 4, wherein said open cavity includes a gel-triggering agent, wherein said gelling agent, upon contact with the gel-triggering agent, causes the wart treatment formulation to form a coherent, soft solid.

9. The system of claim 1, wherein the wart treatment formulation comprises water.

10. The system of claim 1, wherein the substance is water.

11. The system of claim 1, wherein the substance includes a keratolytic agent.

12. The system of claim 1, wherein the substance includes salicylic acid.

13. The system of claim 1, wherein the substance includes urea.

14. The system of claim 1, wherein the substance includes imiquimod.

15. The system of claim 1, wherein the substance is selected from the group of alpha hydroxy acids, sulfur, rescorcinol, urea, benzoyl peroxide, tretinoin, trichloroacetic acid, lactic acid, and mixtures thereof.

16. The system of claim 1, wherein the wart treatment formulation is a gelled oil-in-water emulsion.

17. The system of claim 1, wherein the wart treatment formulation is configured for application to the open cavity such that the open cavity becomes a formulation filled cavity prior to application to the skin.

18. The system of claim 1, wherein the wart treatment formulation is configured for contact with the skin, and the closed cavity is formed by applying an occlusive cover to a patch wall that has been pre-applied to the skin.

19. The system of claim 8, wherein the coherent, soft solid formed upon contacting the gel-triggering agent and the gelling agent leaves substantially no residue on the skin when it is removed.

20. The system of claim 8, wherein the gel-triggering agent is impregnated into an absorbent material disposed within the open cavity.

21. The system of claim 8, wherein the gel-triggering agent is coated onto the adhesive coated substrate of the impermeable cover.

22. The system of claim 8, wherein the gel-triggering agent is coated on the cavity patch within the open cavity.

24. The system of claim 1, wherein the gel-triggering agent is specific for gelling the drug-containing composition when it includes polyvinyl alcohol as the gelling agent.

24. The system of claim 8, wherein the gel-triggering agent is boric acid, a salt of boric acid, or a borate

25. The system of claim 24, wherein the boric acid, the salt of boric acid, or the borate is impregnated into an absorbent material at a quantity from 1 to 20 mg/cm².

26. The system of claim 24, wherein the boric acid or the salt of boric acid is impregnated into an absorbent material at a quantity of from 4 to 8 mg/cm².

27. The system of claim 4, wherein said gelling agent is a polyvinyl alcohol.

28. The system of claim 27, wherein the concentration of the polyvinyl alcohol in the drug-containing composition is from 5% to 40% by weight.

29. The system of claim 27, wherein the concentration of the polyvinyl alcohol in the drug-containing composition is from 8% to 20% by weight.

30. The system of claim 1, wherein the wart treatment formulation comprises an oil phase and an aqueous phase.

31. The system in claim 1, wherein the skin contact region has an area from 0.1 cm² to 20 cm².

32. The system in claim 1, wherein the skin contact region has an area from 0.2 cm² to 5 cm².

33. The system of claim 1, wherein the wart treatment formulation includes a viscosity modifying agent.

34. The system of claim 33, wherein the viscosity modifying agent is selected from the group consisting of polyvinyl alcohol, ethyl cellulose, hydroxy propyl cellulose, a carbomer, a methacrylic polymer, a methacrylate polymer, and combinations thereof.

35. The system of claim 1, wherein the wart treatment formulation comprises imiquimod and water.

36. The system of claim 1, wherein the wart treatment formulation comprises imiquimod and a keratolytic agent.

37. The system of claim 1, wherein the wart treatment formulation comprises water and a keratolytic agent.

38. The system of claim 1, wherein the wart treatment formulation comprises imiquimod, water, and a keratolytic agent.

39. The system of claim 1, wherein the wart treatment formulation comprises a gelling agent and a viscosity modifying agent.

40. The system of claim 1, wherein said wart treatment formulation has a pH greater than 5.

41. The system of claim 1, wherein said wart treatment formulation has a pH from 6 to 9.

42. A method for treating warts, comprising:
enclosing a wart on a skin surface with a cavity patch, said cavity patch comprising an open cavity being configured to become a closed cavity at least in part by application and adherence to the skin surface, said closed cavity containing a wart treatment formulation which is in contact with the wart.

43. The method of claim 42, wherein the open cavity is defined by an occlusive cover and a patch wall attached to the occlusive cover.

44. The method of claim 42, wherein the wart treatment formulation has a viscosity such that it can be conveniently placed into said open cavity without spilling prior to the step of enclosing.

45. The method of claim 43, wherein the wart treatment formulation further includes a gelling agent and the open cavity includes a gel-triggering agent, and wherein that the wart treatment formulation forms a soft, coherent solid after the step of enclosing.

46. The method of claim 42, wherein the wart treatment formulation comprises water.

47. The method of claim 42, wherein the wart treatment substance includes an active ingredient selected from the group consisting of a keratolytic agent, salicylic acid, urea, imiquimod, alpha hydroxy acid, sulfur, rescorcinol, urea, benzoyl peroxide, tretinoin, trichloroacetic acid, lactic acid, and mixtures thereof.

48. The method of claim 42, wherein the wart treatment formulation is a gelled oil-in-water emulsion.

49. The method of claim 42, wherein the step of enclosing includes placing the wart treatment formulation in the open cavity and then applying the wart treatment formulation within the open cavity to the skin.

50. The method of claim 42, wherein the step of enclosing includes applying the cavity patch to the skin, placing the wart treatment formulation within the cavity on the skin, closing the cavity by applying an occlusive cover to a patch wall that has been pre-applied to the skin.

51. The method of claim 45, wherein the gel-triggering agent is impregnated into an absorbent material disposed within the open cavity.

52. The method of claim 45, wherein the gel-triggering agent is coated on walls or an occlusive backing within the open cavity of the cavity patch.

53. The system of claim 45, wherein the gel-triggering agent is specific for gelling the drug-containing composition when it includes polyvinyl alcohol as the gelling agent.

54. The method of claim 45, wherein the gel-triggering agent is boric acid, a salt of boric acid, or a borate.

55. The method of claim 54, wherein the boric acid, the salt of boric acid, or the borate is impregnated into an absorbent material at a quantity from 1 to 20 mg/cm².

56. The method of claim 54, wherein the boric acid or the salt of boric acid is impregnated into an absorbent material at a quantity of from 4 to 8 mg/cm².

57. The method of claim 45, wherein said gelling agent is a polyvinyl alcohol.

58. The method of claim 57, wherein the concentration of the polyvinyl alcohol in the drug-containing composition is from 5% to 40% by weight.

59. The method of claim 57, wherein the concentration of the polyvinyl alcohol in the drug-containing composition is from 8% to 20% by weight.

60. The method of claim 42, wherein the wart treatment formulation comprises an oil phase and an aqueous phase.

61. The method in claim 42, wherein the step of enclosing includes providing contact between the wart treatment composition and a skin contact region having an area from 0.1 cm² to 20 cm².

62. The method in claim 61, wherein the skin contact region has an area from 0.2 cm² to 5 cm².

63. The method of claim 42, wherein the wart treatment formulation includes a viscosity modifying agent.

64. The method of claim 42, wherein the wart treatment formulation comprises at least two of imiquimod, a keratolytic agent, and water.

65. The system of claim 42, wherein said wart treatment formulation has a pH from 6 to 9.
